(19) United States
(12) Patent Application Publication
(10) Pub. No.: US 2024/0360096 A1
(43) Pub. Date: Oct. 31, 2024

(54) CANNABINOIDS FROM SUBSTITUTED RESORCINOL CARBOXYLIC ACIDS

(71) Applicant: LYGOS, INC., Berkeley, CA (US)

(72) Inventors: Nick Ohler, Berkeley, CA (US); Owen Budavich, Berkeley, CA (US); Jason Poulos, Berkeley, CA (US); Camille Indey, Berkeley, CA (US)

(73) Assignee: LYGOS, INC., Berkeley, CA (US)

(21) Appl. No.: 18/553,525

(22) PCT Filed: Apr. 1, 2022
(86) PCT No.: PCT/US22/71494

§ 371 Date: Sep. 29, 2023

Related U.S. Application Data
(60) Provisional application No. 63/170,175, filed on Apr. 2, 2021.
(60) Provisional application No. 63/226,684, filed on Jul. 28, 2021.

(51) Int. Cl. C07D 311/80; C07C 39/23; C07D 311/58

(52) U.S. Cl. CPC C07D 311/80; C07C 39/23; C07D 311/58

(57) ABSTRACT

Provided herein are processes and compositions comprising substituted resorcylic acid or substituted resorcinols.

### CROSS-REFERENCE TO RELATED APPLICATIONS

The present application is a 371 U.S. National Phase Patent Application based on International Application No. PCT/US2020/071494, filed Apr. 1, 2022, which claims the benefit of U.S. Provisional Application No. 63/170,175, filed Apr. 2, 2021 and U.S. Provisional Application No. 63/266,684, filed Jul. 28, 2021, the entire disclosures of which are hereby expressly incorporated herein by reference.

### FIELD

Provided herein are compositions and synthetic processes related to preparing cannabinoids and cannabinoid intermediates thereto.

### BACKGROUND

There is a need for preparing, especially in a cost-effective manner, major and minor cannabinoids.

### SUMMARY

In one aspect, provided herein is a process comprising contacting a compound of Formula I:

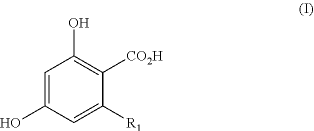

or a salt thereof, wherein R₁ is optionally substituted C₁-C₁₀ alkyl, preferably, C₁-C₈ alkyl, more preferably, n-propyl, n-pentyl, or n-heptyl; optionally substituted C₂-C₁₀ alkenyl; or optionally substituted C₂-C₁₀ alkynyl, with a terpene-based electrophile under conditions suitable to provide a cannabinoid.

Without being bound by theory, the carboxyl group of the resorcinol carboxylic acid of formula 1, acts as a blocking or protecting group for an electrophilically active position of the compound, and reduces or eliminates byproducts. As used herein, an electrophile is an atom or a functional group, which can react with and be replaced by a nucleophile. The reaction and replacement may require activation of the electrophile. Suitable activators include without limitation Lewis acids and Bronsted acids. Suitable nucleophiles include without limitation optionally substituted resorcinol carboxylic acids. Preferred resorcinol carboxylic acids are those utilized herein. Illustrative and non-limiting examples of terpenes and terpene-based electrophiles are included herein.

In another aspect provided herein is a composition comprising:
a cannabinoid, preferably a cannabinoid prepared as provided herein, and
a compound of formula I,
wherein each variable is independently defined as herein.

In another aspect provided herein is a composition comprising:
a terpene-based electrophile, and
a compound of formula I or a salt thereof,
wherein each variable is defined as herein.

In certain embodiments, the compositions provided herein comprise no THC. In certain embodiments, the compositions provided herein comprise less than or equal to 0.3 wt % of THC. In certain embodiments, the compositions provided herein comprise less than or equal to 0.1 wt % of THC. In certain embodiments, the compositions provided herein comprise less than or equal to 0.01 wt % of THC.

### DETAILED DESCRIPTION

### Definition

As used in the specification and claims, the singular form “a,” “an” and “the” include plural references unless the context clearly dictates otherwise.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1, 5, or 10%, e.g., by using the prefix, “about.” It is to be understood, although not always explicitly stated that all numerical designations are preceded by the term “about.” It also is to be understood, although not always explicitly stated, that the reagents described herein are merely exemplary and that equivalents of such are known in the art.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH3-), ethyl (CH3CH2), -n-propyl-(CH3CH2CH2-), isopropyl ((CH3)2CH), -n-butyl- (CH3CH2CH2CH2-), isobutyl ((CH3)2CHCH2-), sec-butyl ((CH3)(CH3CH2)CH), -t-butyl- ((CH3)3C), -n-pentyl-(CH3CH2CH2CH2CH2-), and neopentyl ((CH3)3CCH2-).

“Alkenyl” refers to monovalent straight or branched hydrocarbyl groups having from 2 to 10 carbon atoms and preferably 2 to 6 carbon atoms or preferably 2 to 4 carbon atoms and having at least 1 and preferably from 1 to 2 sites of vinyl (>C═C<) unsaturation. Such groups are exemplified, for example, by vinyl, allyl, and but-3-en-1yl. Included within this term are the cis and trans isomers or mixtures of these isomers.

“Alkynyl” refers to straight or branched monovalent hydrocarbyl groups having from 2 to 10 carbon atoms and preferably 2 to 6 carbon atoms or preferably 2 to 3 carbon atoms and having at least 1 and preferably from 1 to 2 sites of acetylenic (—C≡C—) unsaturation. Examples of such alkynyl groups include ethynyl (—C≡CH), and propargyl (—CH2C≡CH).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO3H, substituted sulfonyl, substituted sulfonyloxy, thioacyl, thiol, alkylthio, and substituted alkylthio, wherein said substituents are as defined herein.

“Heteroalkyl” refers to an alkyl group one or more carbons is replaced with —O—, —S—, SO2, a phosphorous (P) containing moiety, or —NRQ- moieties where RQ is H or C₁-C₆ alkyl. Substituted heteroalkyl refers to a heteroalkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO3H, substituted sulfonyl, substituted sulfonyloxy, thioacyl, thiol, alkylthio, and substituted alkylthio, wherein said substituents are as defined herein.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxyl, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO3H, substituted sulfonyl, substituted sulfonyloxy, thioacyl, thiol, alkylthio, and substituted alkylthio, wherein said substituents are as defined herein and with the proviso that any hydroxyl or thiol substitution is not attached to a vinyl (unsaturated) carbon atom.

Substituents employed herein are such as those defined hereinabove, and without limitation, substituents can be selected from monovalent and divalent groups, such as C1-C10 or C1-C6 alkyl, substituted C1-C10 or C1-C6 alkyl, C2-C6 alkenyl, C2-C6 alkynyl, C6-C10 aryl, C3-C8 cycloalkyl, C2-C10 heterocyclyl, C1-C10 heteroaryl, substituted C2-C6 alkenyl, substituted C2-C6 alkynyl, substituted C6-C10 aryl, substituted C3-C8 cycloalkyl, substituted C2-C10 heterocyclyl, substituted C1-C10 heteroaryl, halo, nitro, cyano, oxo, —CO2H or a C1-C6 alkyl ester thereof.

Unless indicated otherwise, the nomenclature of substituents that are not explicitly defined herein are arrived at by naming the terminal portion of the functionality followed by the adjacent functionality toward the point of attachment. For example, the substituent “alkoxycarbonylalkyl” refers to the group (alkoxy)-C(O)-(alkyl)-.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. In such cases, the maximum number of such substituents is three. That is to say that each of the above definitions is constrained by a limitation that, for example, substituted aryl groups are limited to -substituted aryl-(substituted aryl)-substituted aryl.

It is understood that the above definitions are not intended to include impermissible substitution patterns (e.g., methyl substituted with 5 fluoro groups). Such impermissible substitution patterns are well known to the skilled artisan.

A “salt” is derived from a variety of organic and inorganic counter ions well known in the art and include, when the compound contains an acidic functionality, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate. Salts include acid addition salts formed with inorganic acids or organic acids. Inorganic acids suitable for forming acid addition salts include, by way of example and not limitation, hydrohalide acids (e.g., hydrochloric acid, hydrobromic acid, hydroiodic acid, etc.), sulfuric acid, nitric acid, phosphoric acid, and the like.

Organic acids suitable for forming acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl) benzoic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4-chlorobenzenesulfonic acid, 2-naphthalenesulfonic acid, 4-toluenesulfonic acid, camphorsulfonic acid, etc.), glutamic acid, hydroxynaphthoic-acid, salicylic acid, stearic acid, muconic acid, and the like.

Salts also include salts formed when an acidic proton present in the parent compound is either replaced by a metal ion (e.g., an alkali metal ion, an alkaline earth metal ion, or an aluminum ion) or by an ammonium ion (e.g., an ammonium ion derived from an organic base, such as, ethanolamine, diethanolamine, triethanolamine, morpholine, piperidine, dimethylamine, diethylamine, triethylamine, and ammonia).

### Processes and Compositions

Provided herein is a process comprising contacting a compound of Formula I:

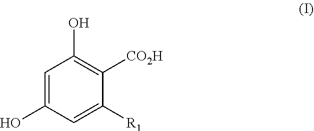

or a salt thereof, wherein R₁ is optionally substituted C₁-C₁₀ alkyl, preferably, C₁-C₈ alkyl, more preferably, n-propyl, n-pentyl, or n-heptyl; optionally substituted C₂-C₁₀ alkenyl; or optionally substituted C₂-C₁₀ alkynyl, with a terpene-based electrophile under conditions suitable to provide a cannabinoid.

Compounds of Formula I are advantaged substrates for the production of cannabinoids, and these compounds are produced efficiently by fermentation as described in Applicant's prior filings. The use of olivetolic acid and similar resorcinol carboxylic acids for synthesis of cannabinoids offers advantages particularly when producing cannabinoids from fermentation-derived substrates. In particular, some of the advantages include (1) minimal processing to prepare the fermentation-derived substrate for synthesis; (2) superior yield during conversion due at least in part to the protection of the ring position containing the carboxylic acid function; (3) ability to produce acidic cannabinoids directly or to produce neutral cannabinoids by decarboxylation after synthesis; and (4) ability to utilize the carboxylic acid function to aid purification processing prior to decarboxylation, when producing neutral cannabinoids. To exemplify (4), since cannabinoids such as tetrahydrocannabinol (THC) and tetrahydrocannabivarin (THCV) are oils, it is advantageous to crystallize their acidic forms for purification, then decarboxylate the purified materials to yield high purity finished products.

In one embodiment, R1 is C1-C10 alkyl. In one embodiment, R1 is C1-C8 alkyl. In one embodiment, R1 is n-propyl. In one embodiment, R1 is n-pentyl. In one embodiment, R1 is n-heptyl. In one embodiment, R1 is 2-phenylethyl (the side chain of perrottetinene). In one embodiment, R1 is C2-C10 alkenyl. In one embodiment, R1 is or C2-C10 alkynyl.

In one embodiment, R1 is substituted C1-C10 alkyl. In one embodiment, R1 is substituted C1-C8 alkyl. In one embodiment, R1 is substituted n-propyl. In one embodiment, R1 is substituted n-pentyl. In one embodiment, R1 is substituted n-heptyl. In one embodiment, R1 is substituted C2-C10 alkenyl. In one embodiment, R1 is or substituted C2-C10 alkynyl. As used herein substituted alkyl includes heteroalkyl and substituted heteroalkyl.

In one embodiment, the terpene-based electrophile is a compound of formula IIA:

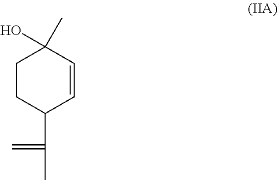

or a diastereomer thereof, or an ester of each thereof. As used in this disclosure, an ester refers to without limitation, a carboxylic acid ester, a sulfonic acid ester, a phosphate ester, and the like. Preferably, the ester is a carboxylic acid ester. In some embodiments, the compound of formula IIA, i.e., the hydroxy form is employed. In some embodiments, the compound of formula IIA is:

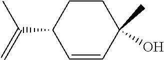

(1R,4R)-4-Isopropenyl-1-methyl-2-cyclohexen-1-ol
or a diastereomer thereof.

In some embodiments, the ester utilized herein is a carboxyl acid ester. Esters with other acids, such as phosphoric or hydrocarbyl sulfonic acids are also contemplated for the process provided herein. The contacting with a compound of formula IIA is performed in presence of a Bronsted acid or Lewis acid catalyst, or may be performed in the presence of a metal, supported metal, or organometallic catalyst, or some combination thereof. In certain embodiments, suitable catalysts include, without limitation, BF3, Sc(OTf)3, ZnCl2, ZnBr2, (p-toluenesulfonic acid) PTSA, (methanesulfonic acid) MsOH, or homogeneous organometallic coupling catalysts. In certain instances, as used herein, aluminum salts and organoaluminum compounds are suitable as Lewis acid catalysts. In some embodiments, the cannabinoid provided upon contacting with a compound of formula IIA is of formula IIIA:

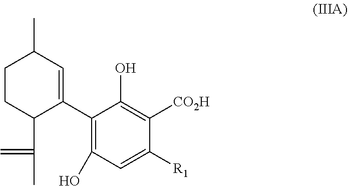

or a delta-8 diastereomer thereof, or a salt of each thereof, wherein R₁ is defined as herein. In some embodiments, the process further comprises decarboxylating the compound of formula IIIA or a delta-8 diastereomer thereof, or a salt of each thereof to provide a compound of formula IVA:

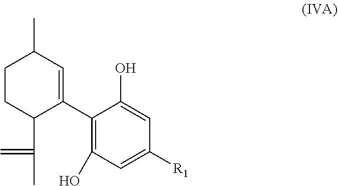

or a delta-8 diastereomer thereof, or a salt of each thereof, wherein R₁ is defined as herein. In some embodiments, the process further comprises cyclizing the compound of formula IIIA or IVA, or a delta-8 diastereomer of each thereof, or a salt of the foregoing, under conditions suitable for cyclization to provide a compound of formula VA or VB:

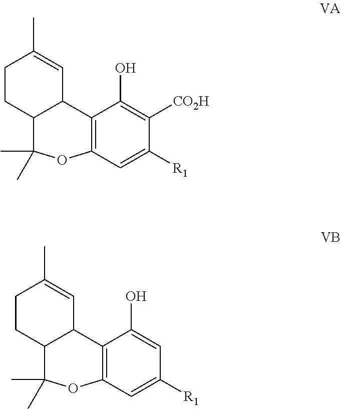

or a delta-8 diastereomer of each thereof, or a salt of the foregoing, wherein R₁ is defined as herein. Cyclization can be performed, e.g., without limitation under Lewis acid catalysis.

In one embodiment, the process comprises reacting a compound of formula IA or a salt thereof with a suitable electrophile, under conditions suitable for cyclization, to provide a compound of formula VA or VB:

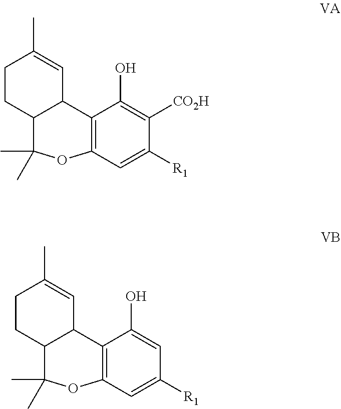

or a delta-8 diastereomer of each thereof, or a salt of the foregoing, wherein R₁ is defined as herein. In one embodiment, the electrophile is a compound of formula IIB (a paramenthenediol):

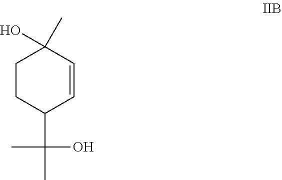

or a diastereomer thereof, or an ester of each thereof. Cyclization can be performed, e.g., without limitation under Lewis acid catalysis.

In one embodiment, the terpene-based electrophile is geraniol, an ester thereof, or a compound where the hydroxy group is converted to a leaving group (collectively, a compound of Formula IIB). In other embodiments, the terpene-based electrophiles include linear sesquiterpenes (C15, such as farnesol) and diterpenes (C20, such as phytane), an ester thereof, or a compound where the hydroxy group is converted to a leaving group. Suitable leaving groups include, without limitation, a halide, hydrocarbyl sulphonic acid esters. The contacting with compound IIB is performed in presence of a Bronsted acid or Lewis acid catalyst, or may be performed in the presence of a metal, supported metal, or organometallic catalyst, or some combination thereof. Suitable catalysts include, without limitation, BF3, Sc(OTf)3, ZnCl2, ZnBr2, (p-toluenesulfonic acid) PTSA, (methanesulfonic acid) MsOH, or homogeneous organometallic coupling catalysts. In some embodiments, the cannabinoid provided upon contacting with a compound of formula IIB is of formula IIIB:

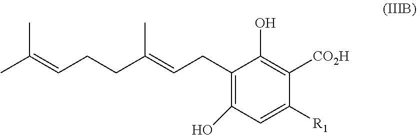

wherein R₁ is defined as herein, or a salt thereof.

In some embodiments, the process further comprises decarboxylating the compound of formula IIIB or a salt thereof to provide a compound of formula IVB:

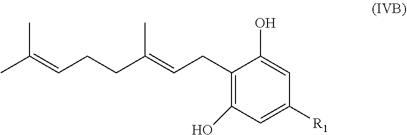

wherein R₁ is defined as herein.

In one embodiment, the terpene-based electrophile is citral. In other embodiments, aldehydes derived from other terpenes such as farnesol is employed as an electrophile. The contacting with citral is performed in presence of a primary hydrocarbyl amine. Suitable examples of primary hydrocarbyl amines include aliphatic primary amines, such as, without limitation tertiary butyl amine. In some embodiments, the cannabinoid provided upon contacting with citral is of formula IIIC:

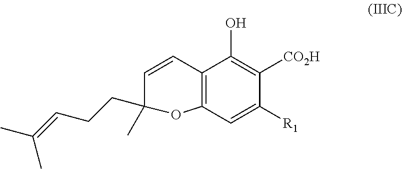

wherein R₁ is defined as herein.
or a salt thereof. In some embodiments, the process further comprises decarboxylating the compound of formula IIIC or a salt thereof to provide a compound of formula IVC:

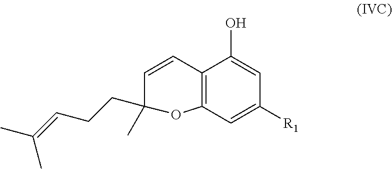

wherein R₁ is defined as herein.

The decarboxylation can be performed, e.g., and without limitation by heating under conditions suitable for decarboxylation.

In one embodiment, the compound reacted is of formula I. In one embodiment, the compound reacted is of formula IIA. In one embodiment, the compound reacted is of formula IIIA. In one embodiment, the compound reacted is of formula IVA. In one embodiment, the compound reacted is of formula VA. In one embodiment, the compound reacted is of formula IIB. In one embodiment, the compound reacted is of formula IIIB. In one embodiment, the compound reacted is of formula IVB. In one embodiment, the compound reacted is of formula VB.

In one embodiment, provided herein is a process wherein the acidic cannabinoid provide herein is purified as such, prior to decarboxylation to produce a neutral cannabinoid finished product. In one embodiment, provided herein is a process wherein the tetrahydrocannabinolic acid (THCA) or a diastereomer produced is purified by chromatography and/or crystallization prior to decarboxylation to yield high-purity THC. In one embodiment, provided herein is a process wherein tetrahydrocannabivarinic acid (THCVA) or a diastereomer thereof is produced by a process of claim 1, and is purified by chromatography and/or crystallization prior to decarboxylation to yield high-purity THCV.

The starting materials are commercially available, e.g., from Sigma Aldrich, Toronto Research Chemicals, etc. as will be well known to the skilled artisan. Alternatively, the compound of formula I is provided by Applicant's process of fermenting glucose and R1CO2H by recombinant Saccharomyces cerevisiae containing Cannabis sativa olivetol synthase (a TKS), olivetolic acid cyclase (a DAB protein), and hexanoyl coA synthetase (a AAE protein) genes. The reactions are preferably carried out in a suitable inert solvent that will be apparent to the skilled artisan upon reading this disclosure, for a sufficient period of time to ensure substantial completion of the reaction as observed, e.g., by thin layer chromatography, high performance liquid chromatography (HPLC), 1H-NMR, etc. If needed to speed up the reaction, the reaction mixture can be heated, as is well known to the skilled artisan. The final and the intermediate compounds are purified, if necessary, by various art known methods such as crystallization, precipitation, column chromatography, and the likes, as will be apparent to the skilled artisan upon reading this disclosure. In some instances, double bond isomers, e.g. and without limitation, delta-9 and delta-9 isomers provided or utilized herein, are separated by column chromatography comprising a stationary phase that comprises silver (Ag+) ion. The purity of a product is determined, e.g. and without limitation by HPLC, optical rotation, NMR, optionally by comparing with an authentic sample, such as a naturally obtained sample. Also, if needed, functional groups may be protected during a reaction, and deprotected as desired. Suitable protecting and deprotecting groups are well known to the skilled artisan, and described, e.g., and without limitation Greene's Protective Groups in Organic Synthesis 4th Edition, by Peter G. M. Wuts and Theodora W. Greene, Wiley; 5th edition (Oct. 27, 2014), incorporated herein by reference.

In another aspect provided herein is a composition comprising:
a compound of formula (IIIA), (IIIB), or (IIIC), or a salt of each thereof and
a compound of formula I or a salt thereof,
wherein each variable is independently defined as herein.

In one embodiment, the composition comprises a compound of formula (IIIA) or a salt thereof. In one embodiment, the composition comprises a compound of formula (IIIB) or a salt thereof. In one embodiment, the composition comprises a compound of formula (IIIC) or a salt thereof.

In another aspect provided herein is a composition comprising:
a compound of formula (IIA) or an ester thereof, (IIB), or citral, and
a compound of formula I or a salt thereof,
wherein each variable is independently defined as herein.

In one embodiment, the composition comprises a compound of formula (IIA). In one embodiment, the composition comprises a compound of formula (IIIB). In one embodiment, the composition comprises citral.

In one aspect, provided herein is a composition of formula VB or a or a delta-8 diastereomer thereof, which is stable for more than about a week, about a month, about 2 months, about 6 months, about 12 months, or more. As used herein stable refers to less than about 20%, preferably less than about 10%, more preferably less than about 5% degradation of the compound of formula VB or a delta-8 diastereomer thereof. In one embodiment, the stable composition is a solution. In another embodiment, the composition is a viscous liquid. In another embodiment, the composition comprises an alkanol, such as without limitation, a C1-C12 alkanol, or a higher alkanol. In another embodiment, the solution comprises a polyol, such as without limitation, a glycerol.

The following example illustrate without limiting the invention or the claims.

### EXAMPLES

### Example I: Production of Cannabidiolic Acid (CBDa)

A resorcinol carboxylic acid, olivetolic acid (8.00 g) and MgSO4 (8.64 g) are mixed with toluene (510 g) and mixed at 20° C. in a jacketed reactor under an N2 atmosphere. A lewis acid, BF3-Et2O (2.8 mL) is added to the olivetolic acid solution. A terpene-based electrophile, p-mentha-2,8-dien-1-ol (PMD, 8.18 g) is diluted in toluene (177.85 g) and slowly added, e.g., over 5 minutes, to the olivetolic acid solution. After about 30 min, the solution is washed with a NaOH aqueous solution (150 mL) twice. The aqueous phases are combined and acidified to about pH 3, e.g., with a citric acid aqueous solution and extracted with toluene (250 mL). The CBDa/toluene solution is concentrated under reduced pressure to an oil. The oil is purified by reverse phase column chromatography with methanol/water as eluent. The purified CBDa is concentrated under reduced pressure to form a purified CBDa concentrate. The purified CBDa concentrate is dissolved in n-heptane and cooled slowly. The product slurry is filtered and washed with cold n-heptane. The product cake is dried to provide purified CBDa crystals (2 g).

### Example II: Production of Cannabidiol (CBD)

A resorcinol carboxylic acid, olivetolic acid (29.0 g), is mixed with toluene (509 g) and tetrahydrofuran (40 g) at −10° C. in a jacketed reactor under an N2 atmosphere. A terpene-based electrophile, p-mentha-2,8-dien-1-ol (PMD, 28.94 g) added to the olivetolic acid solution. A Lewis acid, BF3-Et2O (44 mL) is added to the olivetolic acid solution. After about 2 min, the solution is washed with a NaOH aqueous solution (477 mL) and the phases separated. The CBDa/toluene solution is adjusted to pH 8 with a NaOH aqueous solution and then heated to 95° C. to decarboxylate CBDa to CBD. After 3.5 h, the solution is adjusted to pH 7 with H2SO4 and the aqueous phase is removed. The CBD/toluene solution is concentrated under reduced pressure to an oil. The oil is purified by reverse phase column chromatography with methanol/water as eluent. The purified CBD is concentrated under reduced pressure to form a purified CBD concentrate. The purified CBD concentrate is dissolved in n-heptane and cooled slowly. The product slurry is filtered and washed with cold n-heptane. The product cake is dried to provide purified CBD crystals (15.6 g).

### Example III: Production of Cannabidivarinic Acid (CBDVa)

A resorcinol carboxylic acid, divarinic acid (28.00 g) is mixed with toluene (573 g) and THF (45 g) at −10° C. in a jacketed reactor under an N2 atmosphere. A terpene-based electrophile, p-mentha-2,8-dien-1-ol (PMD, 32.59 g) is added to the divarinic acid solution. A Lewis acid, BF3-Et2O (49 mL) is added to the divarinic acid solution. After about 2 min, the solution is quenched with 318 g of a 10 wt % NaOH solution and the phases separated. The organic phase containing CBDVa is acidified to pH1 with 20 mL 1 M H2SO4 and then concentrated under reduced pressure to an oil. The oil is purified by reverse phase column chromatography with methanol/water as the eluent. The purified CBDVa is concentrated under reduced pressure to form a purified CBDVa concentrate (13.4 g).

### Example IV: Production of Cannabidivarin (CBDV)

A resorcinol carboxylic acid, divarinic acid (28.00 g) is mixed with toluene (573 g) and THF (45 g) at −10° C. in a jacketed reactor under an N2 atmosphere. A terpene-based electrophile, p-mentha-2,8-dien-1-ol (PMD, 32.59 g) is added to the divarinic acid solution. A Lewis acid, BF3-Et2O (49 mL) is added to the divarinic acid solution. After about 2 min, the solution is quenched with 318 g of a 10 wt % NaOH solution and the phases separated. The CBDVa/toluene solution is adjusted to pH 8 with a NaOH aqueous solution and then heated to 95° C. to decarboxylate CBDVa to CBDV. After 3.5 h, the solution is adjusted to pH 7 with H2SO4 and the aqueous phase is removed. The CBDV/toluene solution is concentrated under reduced pressure to an oil. The oil is purified by reverse phase column chromatography with methanol/water as eluent.

### Example V: Production of Tetrahydrocannabivarinic Acid (THCVa)

The purified CBDVa concentrate is mixed in toluene and THF in a 13/1 ratio (0.2 M) under a N2 atmosphere. An acid, previously dried pTSA (through toluene azeotrope) is added to the CBDVa solution and stirred at 25° C. for 66 h. The solution is quenched with saturated NaHCO₃ and the phases separated. The organic phase containing THCVa is acidified and then concentrated under reduced pressure to an oil. The oil is purified by reverse phase column chromatography with methanol/water as the eluent. The purified THCVa is concentrated under reduced pressure to form a purified THCVa concentrate.

### Example VI: Production of Tetrahydrocannabivarin (THCV)

The purified CBDVa concentrate is mixed in toluene and THF in a 13/1 ratio (0.2 M) under a N2 atmosphere. An acid, previously dried pTSA (through toluene azeotrope) is added to the CBDVa solution and stirred at 25° C. for 66 h. The solution is quenched with saturated NaHCO₃ and the phases separated. The THCVa/toluene solution is adjusted to pH 8 with a NaOH aqueous solution and then heated to 95° C. to decarboxylate THCVa to THCV. After 3.5 h, the solution is adjusted to pH 7 with H2SO4 and the aqueous phase is removed. The THCV/toluene solution is concentrated under reduced pressure to an oil. The oil is purified by reverse phase column chromatography with methanol/water as eluent.

### Example VII: Production of THCV from CBDV

CBDV (20.00 g), a compound of formula IVA, is dissolved in o-xylene (0.87 L, 0.1M) in a dry reactor, stirred and heated to 40° C. Triisobutylaluminum 1.0M in hexane (5.2 mL, 7.5 mol %) is then added and the temperature increased to 45° C. and stirred for 6 h. The reaction is quenched slowly with 100 mL of water. The organic phase is separated and filtered to make sure no aluminum oxide is present. The THCV/o-xylene is concentrated under reduce pressure to an oil. The THCV concentrate is purified using reverse phase chromatography with Daisogel-SP-100-10-P stationary phase and 75% methanol mobile phase. The column is rinsed with 100 v % methanol. The high purity THCV eluate is then passed through a HP-20 resin for absorption and then flushed with MeOH. The THCV eluate collected from the MeOH flush is then concentrated under reduced vacuum. A small amount of Ethanol is then added to help stabilize the THCV (11 g isolated at 92% area).

1. A process comprising contacting a compound of Formula I: 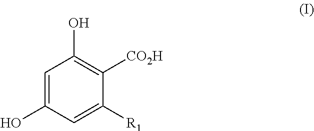
or a salt thereof, wherein R₁ is optionally substituted C₁-C₁₀ alkyl, preferably, C₁-C₈ alkyl, more preferably, n-propyl, n-pentyl, or n-heptyl; optionally substituted C₂-C₁₀ alkenyl; or optionally substituted C₂-C₁₀ alkynyl,
with a terpene-based electrophile under conditions suitable to provide a cannabinoid.

2. The process of claim 1, wherein R₁ is C₁-C₁₀ alkyl, preferably C₁-C₈ alkyl, more preferably n-propyl, n-pentyl, or n-heptyl.

3. The process of claim 1, wherein the terpene-based electrophile is a compound of formula IIA: 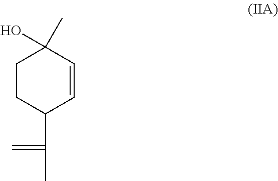
or an ester thereof.

4. The process of claim 1, wherein the terpene-based electrophile is the following, or an ester thereof: 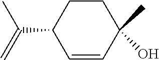
(1R,4R)-4-Isopropenyl-1-methyl-2-cyclohexen-1-ol.

5. The process of claim 3, wherein the is performed in presence of a Bronsted acid or Lewis acid catalyst, or a metal, supported metal, or organometallic catalyst, or some combination thereof.

6. The process of claim 3, wherein the cannabinoid provided is of formula IIIA: 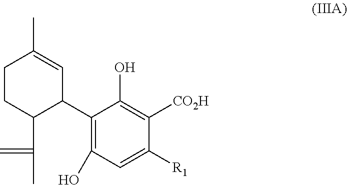
or a salt thereof, wherein R₁ is defined as in claim 1.

7. The process of claim 6, further comprising decarboxylating the compound of formula IIIA or a salt thereof to provide a compound of formula IVA: 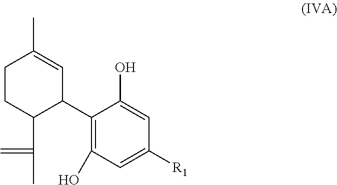
wherein R₁ is defined as in claim 6.

8. The process of claim 1, wherein the terpene-based electrophile is compound IIB, which is geraniol, an ester thereof, or a compound where the hydroxy group is converted to a leaving group.

9. The process of claim 8, wherein the contacting is performed in presence of a Bronsted acid or a Lewis acid catalyst.

10. The process of claim 8, wherein the cannabinoid provided is of formula IIIB: 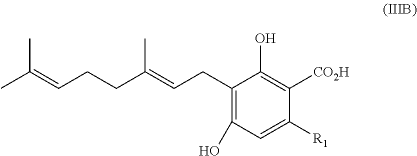
or a salt thereof, wherein R₁ is defined as in claim 1.

11. The process of claim 10, further comprising decarboxylating the compound of formula IIIB or a salt thereof to provide a compound of formula IVB: 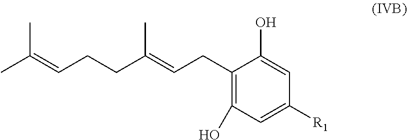
or a salt thereof, wherein R₁ is defined as in claim 10.

12. The process of claim 1, wherein the terpene-based electrophile is citral.

13. The process of claim 12, wherein the contacting is performed in presence of a primary hydrocarbyl amine.

14. The process of claim 13, wherein the cannabinoid provided is of formula IIIC: 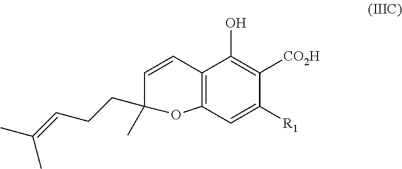
or a salt thereof, wherein R₁ is defined as in claim 1.

15. The process of claim 14, further comprising decarboxylating the compound of formula IIIC or a salt thereof to provide a compound of formula IVC: 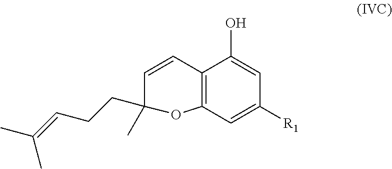
or a salt thereof, wherein R₁ is defined as in claim 14.

16. A composition comprising:
a compound of formula (IIIA), (IIIB), or (IIIC): 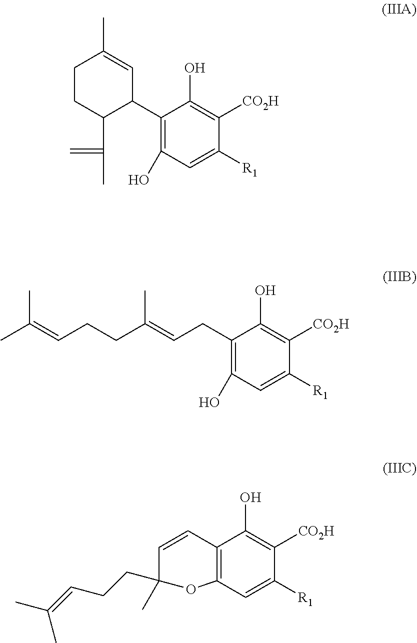
or a salt of each thereof, wherein R₁ is optionally substituted C₁-C₁₀ alkyl, preferably, C₁-C₈ alkyl, more preferably, n-propyl, n-pentyl, or n-heptyl; optionally substituted C₂-C₁₀ alkenyl; or
optionally substituted C₂-C₁₀ alkynyl, and
a compound of formula I: 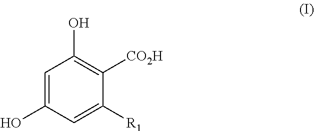
or a salt thereof,
wherein each R₁ is independently defined as above.

17. A composition comprising:
a compound of formula IIA: 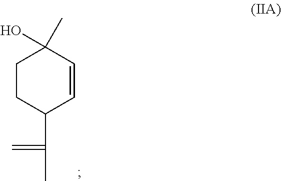
(IIB), which is geraniol, an ester thereof, or a compound where the hydroxy group is converted to a leaving group; or citral, and
a compound of formula I: 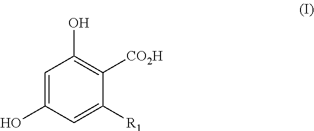
or a salt thereof,
wherein each R₁ is optionally substituted C₁-C₁₀ alkyl, preferably, C₁-C₈ alkyl, more preferably, n-propyl, n-pentyl, or n-heptyl; optionally substituted C₂-C₁₀ alkenyl; or optionally substituted C₂-C₁₀ alkynyl.

18. The process of claim 6 or 7, wherein compound of formula IIIA or IVA is converted on to compounds of formula VA or VB, or a delta-8 diastereomer of each thereof, either within the same reaction process used to produce IIIA and IVA, or in a subsequent reaction process: 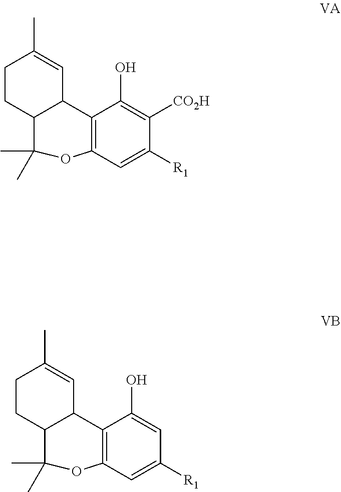
wherein each R₁ independently is defined as in claim 6 or 7.

19. A process according to claim 1 in which the acidic cannabinoid synthesized by the process is purified as such, prior to decarboxylation to produce a neutral cannabinoid finished product.

20. A process according to claim 19 in which tetrahydrocannabiloc acid (THCA) or a diastereomer thereof is produced by a process of claim 1, and is purified by chromatography and/or crystallization prior to decarboxylation to yield high-purity THC.

21. A process according to claim 19 in which tetrahydrocannabivarinic acid (THCVA) or a diastereomer thereof is produced by a process of claim 1, and is purified by chromatography and/or crystallization prior to decarboxylation to yield high-purity THCV.
